(19) United States
(12) Patent Application Publication
(10) Pub. No.: US 2011/0053141 A1
(43) Pub. Date: Mar. 3, 2011

(54) Methods for ultrasensitive detection and quantification of mutant hepatitis B viruses

(75) Inventors: Xiangdong Ren, Malvern, PA (US); Timothy Block, Doylestown, PA (US); Hui Nie, Doyletown, PA (US)

(73) Assignee: INSTITUTE FOR HEPATITIS AND VIRUS RESEARCH, Doylestown, PA (US)

(21) Appl. No.: 12/862,545

(22) Filed: Aug. 24, 2010

Related U.S. Application Data
(60) Provisional application No. 61/236,184, filed on Aug. 24, 2009.

(51) Int. Cl. C12Q 1/70

(52) U.S. Cl. 435/5

(57) ABSTRACT

This invention provides compositions and methods for ultrasensitive detection and quantification of mutant hepatitis B viruses (HBV). The compositions and methods of the invention can be used to detect HBV mutations for diagnostic and prognostic purposes. This invention also provides new application of a TaqMan hydrolysis probe in asymmetric real time PCR and melting curve analysis.

### CROSS-REFERENCE TO RELATED APPLICATIONS

The present application claims the benefit of priority of U.S. Provisional Application No. 61/236184, filed Aug. 24, 2009, which is incorporated by reference.

### BACKGROUND OF THE INVENTION

1. Field of the Invention

The present invention provides methods and reagents for detecting mutant hepatitis B virus (HBV). Thus the invention relates to the fields of medical diagnostics and prognostics as well as the field of molecular biology of nucleotide polymorphism detection.

2. Description of the Prior Art

Chronic hepatitis B (CHB), a chronic disease caused by HBV, is the major cause of liver cirrhosis and hepatocellular carcinoma (HCC) worldwide. An estimated 400 million people worldwide have CHB, and each year, an estimated 500,000 people die of cirrhosis and HCC caused by CHB. It is estimated that approximately 2 million U.S. residents have CHB, and more than $1 billion is spent each year for hepatitis B-related hospitalizations.

HBV is a member of the hepadnaviridae family. It has a approximately 3200 by DNA genome containing four open reading frames for hepatitis B surface antigen (HBsAg), hepatitis B e and core antigens (HBeAg/HBcAg), hepatitis B X protein (HBx), and a viral RNA-dependent DNA polymerase (also named reverse transcriptase). HBeAg is a marker of high infectivity. The HBeAg-negative CHB is a more severe form of disease that is more often associated with cirrhosis and HCC than the HBeAg-positive CHB. Active liver disease is often associated with elevated serum alanine aminotransferase (ALT) (see Locarnini, S. (2005) Semin Liver Dis 25 Suppl 1, 9-19; Hoofnagle, J. H., Doo, E., Liang, T. J., Fleischer, R., and Lok, A. S. (2007) Hepatology 45, 1056-1075; Lai, C. L., and Yuen, M. F. (2007) J Viral Hepat 14 Suppl 1, 6-10; Liaw, Y. F., and Chu, C. M. (2009) Lancet 373, 582-592; Liang, T. J. (2009) Hepatology 49, S13-21, all incorporated by reference).

HBV replication is the key driver of immune-mediated liver injury and disease progression. Use of antiviral drugs to inhibit viral replication can delay or even revert disease progression. Five anti-HBV drugs have been developed; all of them target the viral reverse transcriptase, an enzyme that is essential for viral replication. These drugs are classified as nucleoside analogues (lamivudine, entecavir and telbivudine) and nucleotide analogues (adefovir and tenofovir). Among them, lamivudine is the first orally administered anti-HBV drug approved by FDA in 1998, and it has been used and studied more extensively than other drugs.

One of the major obstacles in successful treatment is the development of drug-resistant viral strains. Take lamivudine as an example, up to 30% of patients may develop drug-resistant mutations at the YMDD (Tyrosine-Methionine-Aspartate-Aspartate) motif of the viral reverse transcriptase (rt) within the first year of treatment. The mutations at codon 204 changes methionine to valine (V) or isoleucine (I), giving rise to rtM204V (YVDD) or rtM204I (YIDD) mutants. Appearance of the YMDD mutants marks the secondary treatment failure because the treatment benefits are diminished (13), and most patients experience flares of ALT elevation, which could be fatal in patients with end stage liver disease.

Resistance to nucleoside analogues shares the rt204 mutations. However, mutations that confer resistance to adefovir and tenofovir are different from each other and do not share the rt204 mutations. Therefore resistance to a nucleoside analogue can be rescued by one of the nucleotide analogues, and vice versa. Although the newer antivirals, such as entecavir and tenofovir, may have lower rates of drug-resistance compared with lamivudine, drug-resistance still poses an issue in therapy monitoring and patient management because treatment is usually required for several years if not life time.

Current clinical practice guidelines recommend that all patients betested for HBV DNA titers prior to treatment and then every 3 months during treatment (see Lok, A. S., Zoulim, F., Locarnini, S., Bartholomeusz, A., Ghany, M. G., Pawlotsky, J. M., Liaw, Y. F., Mizokami, M., and Kuiken, C. (2007) Hepatology 46, 254-265 incorporated by reference). Having detectable HBV DNA at week 24 (6 months) is associated with increased risk of drug resistance. An increase of total HBV DNA by >10 fold from nadir renders diagnosis of virological breakthrough. Due to technical limitations of the current mutation detection methods (see below), HBV mutation detection is only recommended at the time of virological breakthrough to differentiate drug-resistance from non-compliance, and to facilitate selection of an appropriate rescue therapy (Ghany, M. G., and Doo, E. C. (2009) Hepatology 49, S174-184 incorporated by reference). Because virological breakthrough is most often accompanied by a hepatitis flare, mutation detection and change of therapy at this stage will not avoid liver injuries in the patients. This is the major limitation of the current clinical protocol. There is clearly a need for a more sensitive and practical method to monitor and detect the HBV mutant development at an earlier time point so that beneficial clinical decisions can be made promptly.

Direct DNA sequencing is the gold standard for mutation detection because of its highest specificity in nucleotide identification. It is the only method currently used by LabCorp and Quest Diagnostic, two major US diagnostic companies, to detect HBV drug-resistant mutations. However, its sensitivity is low, requiring a minimal mutant/WT ratio of 1:4 (>20% mutant), thus it cannot be used for early detection purpose.

The Line Probe assay, developed by Innogenetics (Ghent, Belgium), offers slightly (4-fold) improved sensitivity (mutant/WT ratio above 1:20, or >5% mutant). Not surprisingly the concordance rate with direct sequencing is >95% (see Hussain, M., Fung, S., Libbrecht, E.,

Sablon, E., Cursaro, C., Andreone, P., and Lok, A. S. (2006) J Clin Microbiol 44, 1094-1097 and Degertekin, B., Hussain, M., Tan, J., Oberhelman, K., and Lok, A. S. (2009) J Hepatol 50, 42-48, both incorporated by reference). Because it is based on hybridization, the results can be less reliable than DNA sequencing. The Line Probe assay for HBV drug resistant mutations is not adopted by Quest Diagnostics, and has recently been removed from the test manual by LabCorp.

Several other methods have been reported for the detection of HBV mutations. They include (1) restriction fragment length polymorphism (RFLP) which relies on restriction enzyme digestion to differentiate the mutant from the wild-type. The readout requires separation of the restriction fragments by gel electrophoresis (Kirishima, T., Okanoue, T., Daimon, Y., Itoh, Y., Nakamura, H., Morita, A., Toyama, T., and Minami, M. (2002) J Hepatol 37, 259-265 and Ohishi, W.,

Shirakawa, H., Kawakami, Y., Kimura, S., Kamiyasu, M., Tazuma, S., Nakanishi, T., and Chayama, K. (2004) J Med Virol 72, 558-565 incorporated by reference); (2) restriction fragment mass polymorphism (RFMP) which uses mass spectrometric analysis to differentiate the mass of restriction fragments (Hong, S. P., Kim, N. K., Hwang, S. G., Chung, H. J., Kim, S., Han, J. H., Kim, H. T., Rim, K. S., Kang, M. S., Yoo, W., and Kim, S. 0. (2004) J Hepatol 40, 837-844; Lee, C. H., Kim, S. O., Byun, K. S., Moon, M. S., Kim, E. O., Yeon, J. E., Yoo, W., and Hong, S. P. (2006) Gastroenterology 130, 1144-1152 and Woo, H. Y., Park, H., Kim, B. I., Jeon, W. K., Cho, Y. K., and Kim, Y. J. (2007) Antivir Ther 12, 7-13 incorporated by reference). Both methods have increased sensitivity than DNA sequencing and Line Probe assay. However, they have limited commercial value due to the involvement of multiple steps that increases the risk of cross-contamination and false positivity. In addition, not all the mutations can be differentiated by a restriction enzyme. Detection of HBV mutation by real time PCR using FRET or TaqMan probe has been reported (Zhang, M., Gong, Y., Osiowy, C., and Minuk, G. Y. (2002) Hepatology 36, 723-728; Pang, A., Yuen, M. F., Yuan, H. J., Lai, C. L., and Kwong, Y. L. (2004) J Hepatol 40, 1008-1017; Shih, Y. H., Yeh, S. H., Chen, P. J., Chou, W. P., Wang, H. Y., Liu, C. J., Lu, S. F., and Chen, D. S. (2008) Antivir Ther 13, 469-480; and Yoshida, S., Hige, S., Yoshida, M., Yamashita, N., Fujisawa, S., Sato, K., Kitamura, T., Nishimura, M., Chuma, M., Asaka, M., and Chiba, H. (2008) Ann Clin Biochem 45, 59-64 incorporated by reference) but the sensitivity is similar to that of the Line Probe assay.

In addition to drug-resistance mutations in the HBV polymerase region, mutations in the precore and the basal core promoter (BCP) regions also have significant clinical importance. The precore mutation, a G to A change at nucleotide 1896 (G1896A), creates a premature stop codon thereby abolishes synthesis of HBeAg (Carman, W. F., Jacyna, M. R., Hadziyannis, S., Karayiannis, P., McGarvey, M. J., Makris, A., and Thomas, H. C. (1989) Lancet 2, 588-591; Akahane, Y., Yamanaka, T., Suzuki, H., Sugai, Y., Tsuda, F., Yotsumoto, S., Omi, S., Okamoto, H., Miyakawa, Y., and Mayumi, M. (1990) Gastroenterology 99, 1113-1119; Brunetto, M. R., Giarin, M. M., Oliveri, F., Chiaberge, E., Baldi, M., Alfarano, A., Serra, A., Saracco, G., Verme, G., Will, H., and et al. (1991) Proc Natl Acad Sci USA 88, 4186-4190, all incorporated by reference). The A1762T/G1764A double mutation in the BCP region diminishes the promoter function and reduces the expression of HBeAg by approximately 50% (Buckwold, V. E., Xu, Z., Chen, M., Yen, T. S., and Ou, J. H. (1996) J Virol 70, 5845-5851; Scaglioni, P. P., Melegari, M., and Wands, J. R. (1997) Virology 233, 374-381; and Tong, S., Kim, K. H., Chante, C., Wands, J., and Li, J. (2005) Int J Med Sci 2, 2-7). Both mutations are associated with HBeAg seroconversion, increased virulence and more severe liver injuries including fulminant hepatitis. BCP mutation is associated with increased risk for developing HCC (35). Ultra-sensitive quantification of precore and BCP mutants may help determine the clinical stage of CHB, predict clinical outcomes such as seroconversion and risk of post-treatment relapse or HCC, and make early intervention possible. The commercially available methods for the detection of HBV precore and BCP mutations also include direct DNA sequencing and Line Probe assays that are qualitative and of low sensitivity. Real time PCR assay has been reported (Zhang, M., Gong, Y., Osiowy, C., and Minuk, G. Y. (2002) Hepatology 36, 723-728 and Pang, A., Yuen, M. F., Yuan, H. J., Lai, C. L., and Kwong, Y. L. (2004) J Hepatol 40, 1008-1017 incorporated by reference), but again there is no significant increase in sensitivity. This is because PCR technology by itself can only detect as low as approximately 5% mutant.

Mutation detection is interfered by the presence of large excess of the wild type DNA. In this invention, we designed and utilized an oligonucleotide to inhibit the amplification of the wild type viral DNA thereby increasing the relative ratio of mutant and the detection sensitivity. This wild type inhibitory oligonucleotide (Wi-oligo) is comprised of modified nucleotide such as the locked nucleic acid (LNA). LNA is a nucleotide analogue that has increased specificity and affinity toward the complementary nucleotide (Singh, S. K., Koshkin, A. A., Wengel, J., and Nielsen, P. (1998) Chem Commun, 455-456 and Hertoghs, K. M., Ellis, J. H., and Catchpole, I. R. (2003) Nucleic Acids Res 31, 5817-5830). Oligonucleotides incorporated with LNAs have been used to increase the mutant detection sensitivity by inhibiting the amplification of the wild-type non-mutated DNA (Nagai, Y., Miyazawa, H., Huqun, Tanaka, T., Udagawa, K., Kato, M., Fukuyama, S., Yokote, A., Kobayashi, K., Kanazawa, M., and Hagiwara, K. (2005) Cancer Res 65, 7276-7282 and Laughlin, T. S., Becker, M. W., Liesveld, J. L., Mulford, D. A., Abboud, C. N., Brown, P., and Rothberg, P. G. (2008) J Mol Diagn 10, 338-345). However, such reports are infrequent because it is often difficult to design such LNA-containing oligonucleotide to effectively suppress the amplification of the wild-type DNA while without affecting the amplification of the mutant. Detection of HBV mutations using LNA-containing oligonucleotide has been recently reported by us with marginal success a (Ren, X. D., Lin, S. Y., Wang, X., Zhou, T., Block, T. M., and Su, Y. H. (2009) J Virol Methods 158, 24-29 incorporated by reference). This invention re-designed the LNA-containing oligonucleotide to have more than 100-fold enhanced inhibitory effect. When combined with DNA sequencing, HBV mutant can now be detected in the presence of 100,000-fold excess of the wild type viral DNA. When combined with real time PCR techniques, it allowed quantification of HBV mutants in the presence of more than 100,000-fold excess of the wild type viral DNA. This invention also includes real time PCR probes for the detection and quantification of HBV mutants.

### SUMMARY OF THE INVENTION

The present invention provides two ultrasensitive systems for the detection of HBV mutants. One is a qualitative PCR-based mutation detection system exemplified by DNA sequencing; the other is a quantitative mutant detection system exemplified by a 2-stage real time PCR. Both methods relied on the use of a locked nucleic acid (LNA)-containing oligonucleotide that can efficiently suppress the amplification of wild-type HBV DNA without affecting the amplification of the HBV mutants.

### BRIEF DESCRIPTION OF THE DRAWING

FIG. 1. Schematic representation of the wild type inhibitory PCR reaction. Shown are (A) displacement of the Primer A on the wild type template sequence; (B) no displacement on the mutant DNA template; and (C) effect of the Wi-oligo is graphically shown using the fluorescent intensity of SYBR green dye.

FIG. 2. Schematic of the TaqMan hydrolysis having a 5′-fluorescence label and a 3′-quencher.

### DETAILED DESCRIPTION OF THE INVENTION

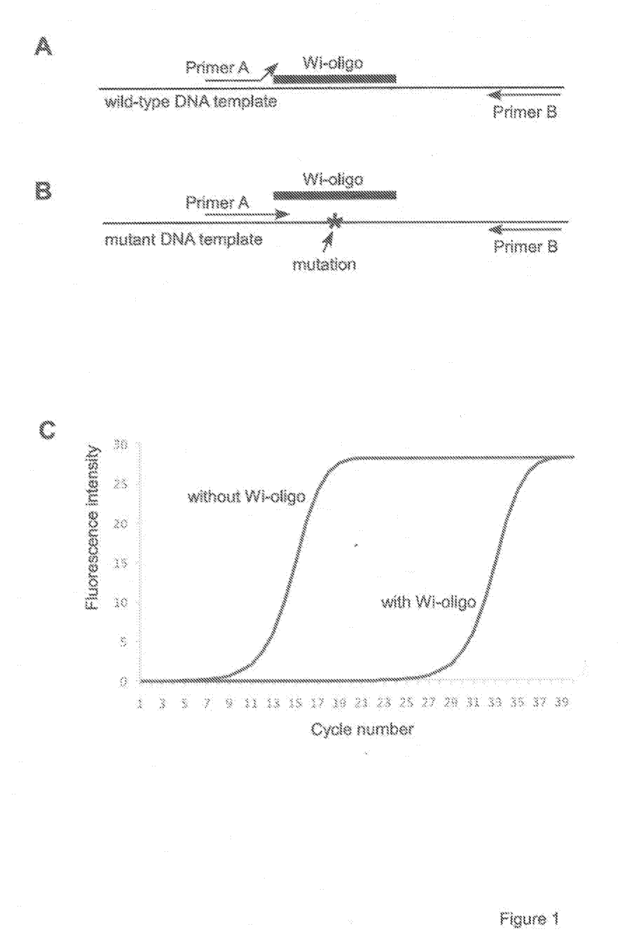

Wild-type inhibitory oligonucleotides (Wi-oligos) are developed to inhibit the amplification of the wild type HBV DNA in PCR, and to selectively amplify the HBV mutants. Referring now to FIG. 1, there is shown the Wi-oligo spans the region of DNA sequence where mutation of interest occurs. The Wi-oligo has more than 80% sequence identity to the target HBV sequence or the complementary strand of HBV sequence. At the mutation site, the Wi-oligo has perfect match to the wild type sequence, and has a mismatch or mismatches to the mutant DNA sequence due to the presence of mutation(s). The Wi-oligo has up to 50 nucleotides, and contains at least one LNA nucleotide. The Wi-oligo is phosphorylated at the 3′-end so that it will not function as a primer.

In further detail, still referring to the invention of FIG. 1, one of the PCR amplification primers partially overlaps with the Wi-oligo, and is in the same direction as the Wi-oligo. This primer is designated as the Primer A. The other amplification primer is designated as the Primer B. The Primer A in this invention does not cover the mutation site thus will allow mutation detection by downstream applications such as DNA sequencing or real time PCR. Primer A can have up to 50 nucleotides with more than 80% sequence identity to the target sequence. The overlap between the Primer A and the Wi-oligo is at least one nucleotide. Primer A and Primer B may or may not contain LNA nucleotides. During thermal cycling, the Wi-oligo will be able to bind to the wild type sequence strongly due to the presence of the LNA nucleotide(s), but bind to the mutant sequence weakly due to the mismatch(es). This results in displacement of the Primer A on the wild type template sequence (FIG. 1A), but not so on the mutant DNA template (FIG. 1B). This in turn results in inhibition of PCR amplification of the wild type HBV DNA, but allows amplification of the mutant HBV DNA. In further detail, still referring to the invention of FIG. 1, the effect of the Wi-oligo is visualized with the aid of SYBR green dye in the PCR (FIG. 1C). In the presence of the Wi-oligo, the wild type DNA is amplified less efficiently, as indicated by a delayed (right-shifted) amplification curve compared with the amplification in the absence of the Wi-oligo. The ultra-sensitive wild type-inhibitory direct DNA sequencing method for HBV mutation detection is comprised of a wild-type inhibitory PCR (Wi-PCR) followed by DNA sequencing. The Wi-PCR is performed by adding the inhibitory oligonucleotide (the Wi-oligo) to the otherwise regular PCR reaction that contains the amplification primers (Primers A and B), DNA polymerase, the polymerase buffer and dNTPs. The concentration of the Primer A and B can be in the range of 0.1 to 1 μM. The concentration of the Wi-oligo can be in the range of 0.2 to 50 μM. The thermal profile may or may not include an oligonucleotide-binding step in between the denaturation and annealing steps. The Wi-PCR can be in between 10 and 55 cycles depending on the downstream applications. For DNA sequencing as the downstream application, Wi-PCR is performed for 25-55 cycles until sufficient amount of DNA is generated. The PCR product is purified to remove the free primers, and is subjected to DNA sequencing using the Primer B. To distinguish from the regular direct DNA sequencing, we name it Wi-direct DNA sequencing for wild type inhibitory direct DNA sequencing.

In addition to direct sequencing, the Wi-PCR can be followed by any other mutation detection methods, either qualitative or quantitative, to significantly increase the mutation detection sensitivity. These methods may include, but not limited to, solid phase hybridization (for example, Southern blotting and dot blotting), liquid phase hybridization (such as melting curve analysis), reverse hybridization (labeled PCR products hybridizing to the immobilized oligonucleotides), mass spectrometer, and real time PCR.

The ultra-sensitive quantitative HBV mutation detection system is comprised of a Wi-PCR followed by a real time PCR using a fluorescence-labeled oligonucleotide probe. This real time PCR can be, but not limited to, a TaqMan PCR using a hydrolysis probe, a FRET PCR, a SimpleProbe PCR, or a Scorpion probe PCR. The Wi-PCR is performed for 10-20 cycles, followed by 30-40 cycles of real time PCR. This is designated as Wi-quantitative PCR or Wi-qPCR.

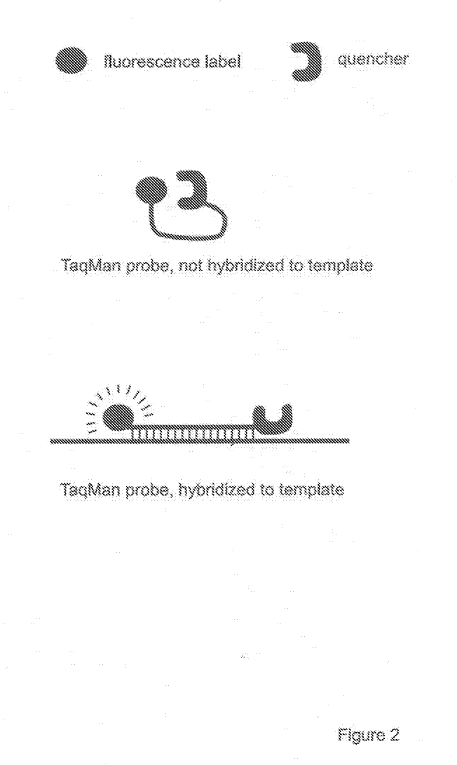

Referring now to FIG. 2, there is shown a new application of the TaqMan hydrolysis PCR probe. A TaqMan probe has a 5′-fluorescence label and a 3′-quencher. Fluorescence emission is the lowest when the probe is not hybridized to the template because the fluorescence group has the shortest distance to the quencher. The fluorescence emission increases when the probe is hybridized to the template, but has the highest signal when the fluorescence group is hydrolyzed and is further away from the quencher. During the TaqMan PCR, primer extension leads to hydrolysis of the 5′-nucleotide, releasing the fluorescence group. This is achieved by the 5′-3′ exonuclease activity of the Taq polymerase. When a 5′-3′ exo-minus Taq polymerase is used, however, the fluorescence labeled probe will not be hydrolyzed. Now the TaqMan probe can be used similarly as a SimpleProbe or a scorpion probe in an asymmetric PCR where the concentration of the Primer B is 5-10 fold higher than that of the Primer A. Specifically, the TaqMan probe can now be used to quantify PCR amplification in real time by means of its fluorescence intensity change after binding to the amplified PCR products rather than by the hydrolysis of the probe. Because the TaqMan probe is preserved during the real time PCR, it can be used for melting curve analysis following PCR amplification. Unlike the SimpleProbe which has only one color (fluorescein), the TaqMan probes can be labeled with different fluorescence dyes, thus allowing multiplex amplification and melting curve analysis. The price of a TaqMan probe is generally much cheaper than a SimpleProbe or a Scorpion probe. An example is given below.

### EXAMPLES

### Example 1

Wi-PCR for HBV rt204 codon. The Wi-oligo, and the amplification primers A and B are 5′-tggattcagtTAtATGGAtgat-PH, 5′-ccccactgtttggctttcagttat-3′ and 5′-gcggtcgggtaaccccatctttttgtttt-3′, respectively. Capital letters indicate LNA nucleotides. “-PH” stands for 3′-end phosphorylation. The Wi-PCR is carried out using the hot-start Taq polymerase (Roche) or hot-start PFU polymerase (Agilent), the appropriate PCR buffer, 200 μM dNTP, 0.5 μM each of the amplification primers, 2 μM of inhibitory oligonucleotide, and the template DNA. The thermal profile is 95° C. for 2-10 min to activate the polymerase, followed by 18 cycles (for downstream qPCR) or 45 cycles (for DNA sequencing) of 95° C. 10 seconds, 76° C. 20 seconds, 60° C. 10 seconds and 62° C. 15 seconds. Amplification of the wild type HBV DNA is inhibited by 12.7 cycles or about 5000 fold, while amplification of the mutant (the GTG variant) is reduced by only 0.39 cycle.

### Example 2

Wi-PCR for the HBV BCP region. The Wi-oligo, and the amplification primers A and B are 5′-aggagattaGgttAaAGGtctttGt-PH, 5′-gaggagttgggggaggagattaggttaa-3′ and 5′-gaagtggtgttcaatttatgcctacagcctccta-3′, respectively. Capital letters indicate. LNA nucleotides. “-PH” stands for 3′-end phosphorylation. The Wi-PCR is carried out using the hot-start DNA polymerase, 200 μM dNTP, 0.5 μM each of the amplification primers, 2 μM of inhibitory oligonucleotide, and the template DNA. The thermal profile is 95° C. for 2-10 min to activate the polymerase, followed by 18 cycles (for downstream qPCR) or 45 cycles (for DNA sequencing) of 95° C. 10 seconds, 75° C. 15 seconds, 57° C. 10 seconds and 65° C. 5 seconds. Amplification of the wild type HBV DNA is inhibited by 12.68 cycles or about 7000 fold, while amplification of the mutant (1762T/1764A) is reduced by only 0.24 cycle.

### Example 3

Wi-PCR for the precore region of HBV genome. The Wi-oligo, and the amplification primers A and B are 5'-gtccatgCcCCAAagcc-PH, 5′-tccaaattctttataagggtcaatgtccatg-3′ and 5′-cctccaagctgtgccttgg-3′ , respectively. Capital letters indicate LNA nucleotides. “-PH” stands for 3′-end phosphorylation. The Wi-PCR is carried out using the hot-start DNA polymerase, 200 μM dNTP, 0.5 μM each of the amplification primers, 2 μM of inhibitory oligonucleotide, and the template DNA. The thermal profile is 95° C. for 2-10 min to activate the polymerase, followed by 18 cycles (for downstream qPCR) or 45 cycles (for DNA sequencing) of 95° C. 10 seconds, 79° C. 20 seconds and 59° C. 10 seconds. Amplification of the wild type HBV DNA is inhibited by ˜14 cycles or about 16,000 fold, while amplification of the mutant (1896A) is reduced by only ˜0.8 cycle.

### Example 4

Wi-qPCR for HBV rt204 codon mutations. One microliter of the Wi-PCR reaction performed for 18 cycles as described above, is added to a PCR reaction which contains Genotyping Master Mix (Roche), 5 mM MgCl2, 0.1 μM forward primer (5′-ccccactgtttggctttcagttat-3′), 0.5 uM reverse primer (5′-gcggtcgggtaaccccatctttttgtttt-3′), and 0.1 uM SimpleProbe (5′-tggctIXttcagttaTGTTGa-PH). The SimpleProbe is internally labeled (IXt) and contains LNA nucleotides (capital letters). Amplification is performed by 37 cycles of 95° C. 10 seconds, 55° C. 10 seconds with fluorescence detection, and 72° C. 5 seconds. Immediately after the amplification, a melting curve analysis is performed at a temperature range of 35-80° C. The melting temperatures of this SimpleProbe to the ATF, GTG, ATA, ATC, ATG (wild type) are 57.8, 55, 52, 50.5 and 49.2° C., respectively. The same probe can be used to quantify the GTG and ATT variants of mutants using the above described thermal profile. To be able to quantify the amount of mutant, serial diluted plasmids carrying the mutant (GTG variant) are included in the Wi-PCR and further amplified in the real time PCR.

### Example 5

Wi-qPCR for the HBV BCP region. One microliter of the Wi-PCR reaction performed for 18 cycles as described above, is added to a PCR reaction which contains Genotyping Master Mix (Roche), 2 mM MgCl2, 0.1 uM forward primer (5′-gataagttgaggagttggggg-3′), 0.5 uM reverse primer (5′-gcggtcgggtaaccccatctttttgtttt-3′), and 0.1 uM SimpleProbe (5′-ggagaIXttaGgttAaTGAtct-PH). The SimpleProbe is internally labeled (IXt) and contains LNA nucleotides (capital letters). Amplification is performed by 37 cycles of 95° C. 10 seconds, 55° C. 10 seconds with fluorescence detection, and 72° C. 5 seconds. Immediately after the amplification, a melting curve analysis is performed at a temperature range of 30-75° C. In the melting curve, the wild type has a melting temperature of about 49° C., while the 1762T/1764A mutant has a melting temperature of 62° C. To be able to quantify the amount of mutant, serial diluted plasmids carrying the 1762T/1764A mutant are included in the Wi-PCR and further amplified in the real time PCR.

### Example 6

Wi-qPCR for the precore region of HBV genome. One microliter of the Wi-PCR reaction performed for 18 cycles as described above, is added to a PCR reaction which contains Genotyping Master Mix (Roche), 3 mM MgCl2, 0.1 uM forward primer (5′-gaagctccaaattctttataagggtcaatgtccatg-3′), 0.5 uM reverse primer (5′-cctccaagctgtgcc-3′), and 0.1 uM SimpleProbe (5′-gtcaIXatgtccatgTcCTAaagcc-PH). The SimpleProbe is internally labeled (IXa) and contains LNA nucleotides (capital letters). Amplification is performed by 37 cycles of 95° C. 10 seconds, 66° C. 10 seconds with fluorescence detection, and 72° C. 5 seconds. Immediately after the amplification, a melting curve analysis is performed at a temperature range of 40-85° C. The melting temperatures of the wild type, the 1896A mutant and the 1896A/1899A mutant are 61° C., 67° C. and 70° C., respectively. To be able to quantify the amount of mutant, serial diluted plasmids carrying the 1896A mutant are included in the Wi-PCR and further amplified in the real time PCR.

### Example 7

Use of a TaqMan probe for quantification and melting curve analysis in asymmetric real time PCR. To quantify the ATA variant of HBV rt204 codon, a TaqMan probe is developed as 5′-Cy5-tcagttataTAGa-quencher, where Cy5 is a fluorescent label, and the capital letters (TAG) represent LNA nucleotides. When the PCR is performed using a 5′-3′ exo-minus Taq polymerase, 5 mM. MgCl2, 0.1 uM forward primer (5′-ccccactgtttggctttcagttat-3′), 0.5 uM reverse primer (5′-gcggtcgggtaaccccatcatttgatt-3′), and 0.1 uM of the ATA TaqMan probe with a thermal cycle profile of 95° C. 10 seconds, 55° C. 10 seconds with fluorescence detection, and 72° C. 5 seconds, only the ATA variant of rt204 codon is detected during amplification (FIG. 2B). io Melting curve (FIG. 2C) shows a melting peak of ATA that is distinct from the wild type (ATG) and the other variants (ATT, ATC and GTG).

### Example 8

Use of a TaqMan probe for melting curve analysis in asymmetric PCR. To differentiate between 1896A and 1899A precore mutations, a TaqMan probe is designed as 5′-FAM-tccatgccctaaagcc-Quencher. The PCR is performed using a 5′-3′ exo-minus Taq polymerase, 3 mM MgCl2, 0.1 uM forward primer (5′-gaagctccaaattctttataagggtcaatgtccatg-3′), 0.5 uM reverse primer (5′-cctccaagctgtgcc-3′), and 0.1 uM of the probe. Amplification is performed by 37 cycles of 95° C. 10 seconds, 66° C. 10 seconds with fluorescence detection, and 72° C. 5 seconds. Immediately after the amplification, a melting curve analysis is performed at a temperature range of 40-85° C. The melting temperatures of the 1896A and 1899A mutants are 62° C., and 41° C., respectively.

The advantages of the present invention include, without limitation, that it enables detection and quantification of HBV mutations with an extraordinarily high sensitivity. In broad embodiment, the present invention can be applied to the detection of other genetic mutations with ultra-high sensitivity. The use of TaqMan probe for melting curve analysis in asymmetric PCR using a 5′-3′ exo-minus Taq polymerase allows multiplex melting curve analysis at a much lower cost.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. An ultra-sensitive method for HBV mutation detection comprising:
a. inhibiting the wild type HBV by PCR wherein said PCR reaction comprises Primers A and B, DNA polymerase, polymerase buffer and dNTPs to produce a PCR product; and
b. determining the HBV mutation from said PCR product.

2. The method of claim 1 wherein said determining is by sequence analysis.

3. The method of claim 1 wherein said inhibition is the addition of inhibitory oligonucleotides to the PCR reaction.

4. The method of claim 3 wherein said inhibitory oligonucleotides are in a concentration range of 0.2 to 50 micromolar.

5. The method of claim 1 wherein said Primer A and Primer B are in a concentration range of 0.1 to 1.0 micromolar.

6. The method of claim 1 wherein said PCR reaction is between 15 and 55 cycles.

7. The method of claim 1 wherein said PCR product is purified to remove free primers.

8. The method of claim 7 wherein said PCR product is further sequenced using Primer B.

9. The method of claim 1 wherein said determining HBV mutation is a method selected from a group consisting of solid phase hybridization, Southern blotting, dot blotting, liquid phase hybridization, reverse hybridization, mass spectrometry, and real time PCR.

10. An ultra-sensitive HBV mutation quantification system comprising:
a. inhibiting the wild type HBV by PCR wherein said PCR reaction comprises Primers A and B, DNA polymerase, polymerase buffer and dNTPs; and
b. quantifying the HBV mutation using real time PCR.

11. The method of claim 10 wherein fluorescent-labeled oligonucleotide probes are used in said real time PCR.

12. The method of claim 10 wherein said real time PCR includes the use of a TaqMan hydrolysis PCR probe having a 5′-fluorescence label and a′3′-quencher.
